(19) United States
(12) Patent Application Publication
(10) Pub. No.: US 2005/0049661 A1
(43) Pub. Date: Mar. 3, 2005

(54) Ice belt to reduce body temperature

(76) Inventor: Shirley B. Koffroth, Palmdale, CA (US)

Correspondence Address: Mrs. Shirley B. Koffroth, 1342 Jaguar Court, Palmdale, CA, 93551, US

(21) Appl. No.: 10/816,420

(22) Filed: Apr. 1, 2004

Related U.S. Application Data
(60) Provisional application No. 60/499,930, filed on Sep. 3, 2003.

(51) Int. Cl. A61F 7/00; A61F 7/12

(52) U.S. Cl. 607/108; 607/112; 607/114

(57) ABSTRACT

The Ice Belt To Reduce Body Temperature is a belt made of a turned out strip of flexible material of amble width and length to gird the waist from the midriff down, contains pocket-like receptacles on its innermost body-contacting side for the placement of flexible ice bags, and is secured around the waist by a connecting means. The Ice Belt to Reduce Body Temperature is a new concept of belts intended for sports, exercise and safety because the Ice Belt To Reduce Body Temperature provides the wearer with the comfort of a lower body temperature when exposed to high climatic conditions. In fact, most belts pertaining to the prior art are designed to promote sweating—a symptom of high body temperature—rather than reduce sweating. Therefore, the Ice Belt helps to lessen the risk of the user acquiring heat exhaustion when exposed to high climatic conditions.

### CROSS-REFERENCE TO RELATED APPLICATION:

This is a Non-Provisional patent application claiming the benefit of U.S. Provisional Patent Application Serial No. 60/499,930 filed Sept. 9, 2003, which is hereby incorporated by reference in its entirety.

### STATEMENT REGARDING FED SPONSORED R & D:

There is no sponsoring.

### SEQUENCE LISTING A TABLE OR A COMPUTER PROGRAM APPENDIX

None

### DESCRIPTION

### BACKGROUND OF THE INVENTION

None of the prior art belts designed for sports, exercise or safety provide the wearer with the comfort of a lower body temperature when exposed to high climatic temperatures. In fact, most prior art belts are designed to promote sweating—a symptom of high body temperature—rather than reduce sweating. Therefore, the Ice Belt to reduce body temperature—from here on referred to as the Ice Belt—far surpasses prior art belts intended for sports, exercise and safety. Why? Because the Ice Belt's principal utility is to lower body temperature and not elevate it; thus avoiding that the user of the Ice Belt is vulnerable to the dangerous condition of heat exhaustion.

### BRIEF SUMMARY OF THE INVENTION

With the above in mind, I contend that my invention, an Ice Belt

### BRIEF SUMMARY OF THE INVENTION

(continued from page 1) to reduce body temperature, is unique because the user acquires an elevated degree of resistance to high climatic temperatures and therefore his body functions operate more normally. Thus, the principal object of my invention is to provide a more comfortable body temperature for the user when exposed to high temperatures, thus elevating his resistance to heavy heat as found in tropical zones. Therefore, the Ice Belt's principal utility is to lower the body temperature and help prevent heat exhaustion.

### BRIEF DESCRIPTION OF THE DRAWINGS

FIG. 1 is a perspective view of the Ice Belt in extended condition showing all of it's components and drawn to a reduced scale.

FIG. 2 is a perspective view of the Ice Belt in curled configuration. The drawing is approximately the same scale as FIG. 1.

FIG. 3 is a front view and shows the Ice Belt fastened onto a dummy figure. FIG. 3 is drawn in a similar scale as FIGS. 1 & 2.

FIG. 4 is a back view and shows the Ice Belt fastened onto a dummy figure. FIG. 4 is drawn similar to the scales used in

### DESCRIPTION OF THE PREFERRED EMBODIMENTS

Similar parts are designated by the same reference characters in the several views of my invention, the Ice Belt To Reduce Body Temperature, from now on referred to as, the Ice Belt.

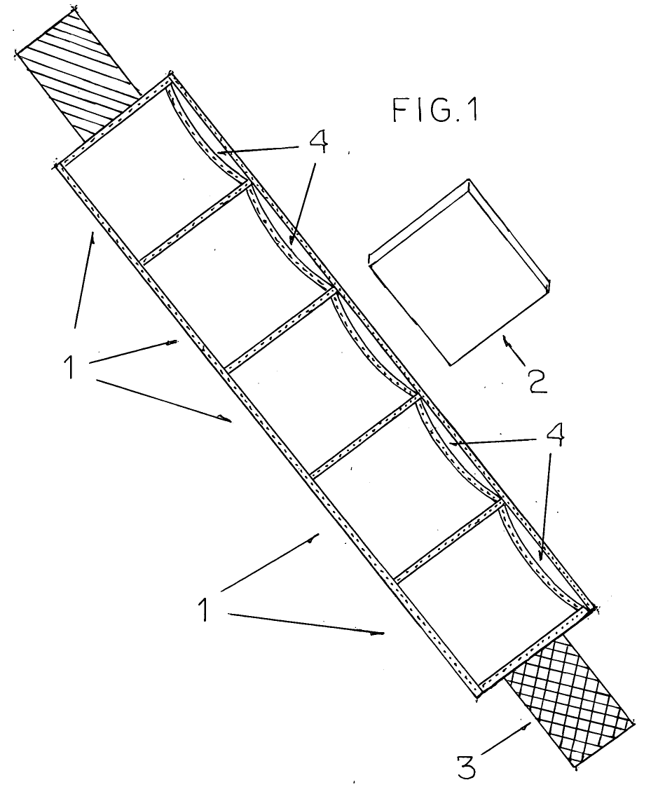

This invention, the Ice Belt, FIG. 1, incorporates the use of pocket-like receptacles, Ref. 1, for the insertion of flexible bags, Ref. 2, made of plastic and containing frozen blue or treated ice. The Ice Belt is attached at the ends by mating fasteners of Velcro, Ref. 3. The pocket-like receptacles, Ref. 1, contain a connecting means placed on the inner-top side, Ref. 4, of the pocket-like receptacles to secure the pocket.

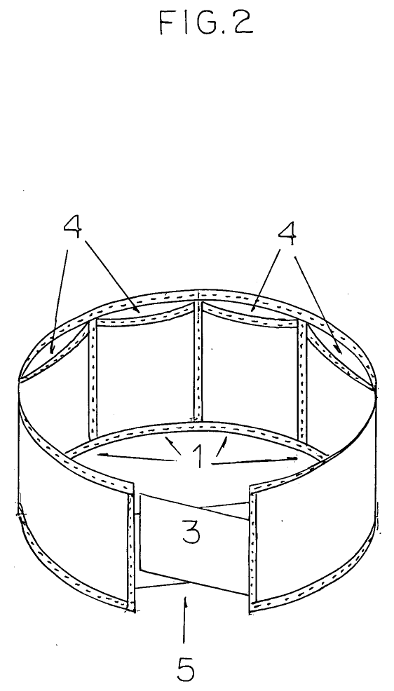

FIG. 2 is a perspective view of the Ice Belt in curled configuration. When the Ice belt is closed as shown in FIG. 2, the pockets containing the ice bags are located to the inner side of the belt, Ref. 1. FIG. 2 also shows the Velcro fasteners, Ref.3, located on the belt's extremities, closed to their tightest position as the fasteners can be accommodated to a loose or tightened position, Ref. 5, depending on changes in the waistband measurements.

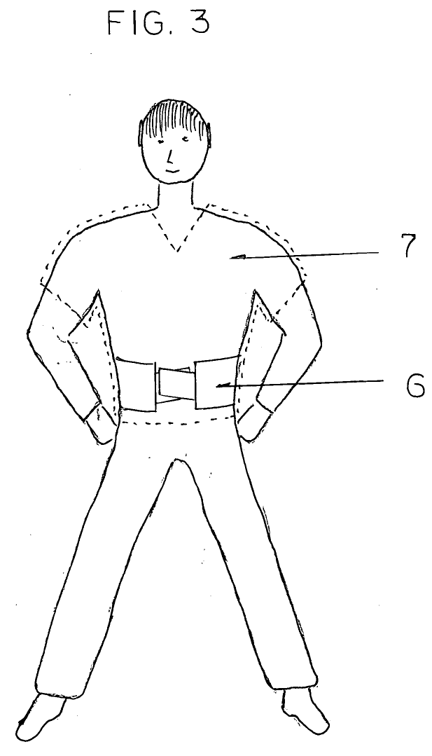

FIG. 3 is a front view and shows the Ice Belt, Ref. 6, fastened from the dummy figure's midriff down to his waist. The drawing in FIG. 3 also depicts the Ice Belt, Ref. 6, as shown underneath a body garment; in this case, a shirt, Ref. 7.

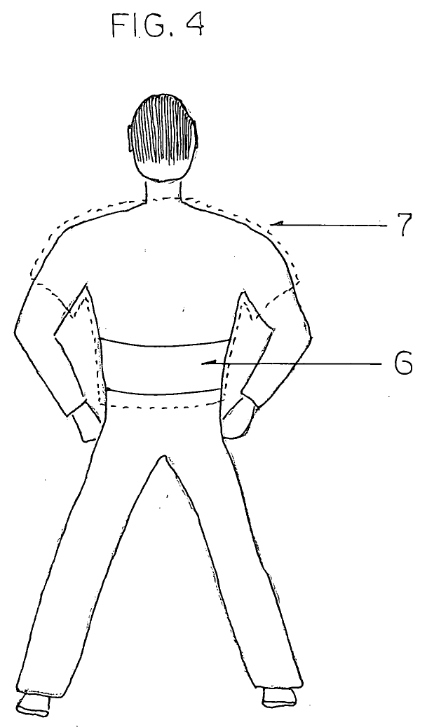

FIG. 4 shows the Ice Belt, Ref. 6, from behind as it looks when positioned on the wearer. The drawing in FIG. 4 also depicts the Ice Belt, Ref. 6, as shown underneath a body garment; in this case, a shirt, Ref. 7.

### DETAILED DESCRIPTION OF THE INVENTION

The invention—an Ice belt to reduce body temperature—consists of a strip of flexible material made of any pliable fabric approximately 4 to 5 inches wide and the length depending on the user's waist measurement. The Ice Belt is equipped with pocket-like receptacle sewn to the strip of flexible material on the innermost body-contacting side of the Ice Belt; the pocket-like receptacle's function is to hold flexible bags of blue or treated ice. The Ice Belt is designed to fasten around the midriff and extend to the waist by means of an ample length of Velcro sewn to each extremity of the Ice Belt. These Velcro fasteners on both ends of the Ice Belt's extremities should be of sufficient length for accommodation of normal changes in the waistband measurement. The above because a person's waistband measurement is not constant. It basically falls into four different measurement length classifications: standing, before eating; standing, after eating; sitting, before eating and sitting after eating. Also there is one more variable regarding waistband measurement common to each of the four measurement classifications, and this is the effect on waist measurement length caused from breathing, both inhaling and exhaling.

By the use of extended lengths of Velcro sewn to the Ice Belt's extremities; the Ice Belt can accommodate to expanding or disbanding conditions of the waistband simply by opening the Velcro fasteners sewn to the Ice Belt's extremities and readjusting the fasteners; thus accommodating the Ice Belt to a slightly smaller or larger waistband measurement.

Plus this suggested facility of extended lengths of Velcro for hand adjustment to waistband changes, the Ice Belt should be fabricated in various sizes; small, medium, large, extra large etc; the measurements depending on whether the Ice Belt is intended for a child, adolescent, man or woman, heavy or thin. The Ice Belt is more effective when worn underneath ones clothing with the pocket-like receptacles that are attached to the innermost body-contacting side of the Ice Belt in direct contact with the user's body.

The Ice Belt's front piece fabrication is very simple as it consists of sewing together two long strips of flexible material cut to allow hemming for a turned out measurement of from 4 to 5 inches in width, and the length depending on the desired waist measurement. The ends of the strip of flexible materials should be left open for the insertion of the Velcro fasteners; once the two sewn strips of flexible material have been turned out, the Velcro fasteners are sewn to both of the Ice Belt's extremities. Then the securing method(snaps or Velcro etc.)are placed to the inner-top side, Ref. 4, of the pocket-like receptacles. The strip of pockets, Ref. 1, is then secured to the Ice Belt's front piece section, by sewing it into place on the bottom and sides of the turned out strip of flexible material to its innermost body contacting side. The ice bags, Ref. 2, are frozen before placed inside the Ice Belt's receptacles. When the ice in the ice bags melts—depending on the temperature, body functions, and outer garment—used bags can be replaced by freshly frozen ones, or the Ice Belt in its entity can be put into a freezer compartment until the liquid in the bags contained in the Ice Belt's pockets has frozen again. In a tempetature from about 85 F. to 95 F., the ice should stay in tack for about 2 to 3 hours, depending on body exertion and the outer garment. In higher temperatures, the ice may only last for an hour; also depending on the outer garment and the exertion used while wearing the Ice Belt. If worn under a bullet proof vest or an armored shield as used for Martial Arts—both insulating devices—the ice's duration can be doubled.

The present embodiments of this invention are to be considered in all respects as descriptive and illustrative, but not restrictive; the scope of the invention being indicated by the appended claims rather than by the foregoing description; therefore, any changes considered more functional to the original design and utility of my invention, the Ice Belt to Reduce Body Temperature, as long as they come within the meaning and range of my invention's basic conception, a device to lower body temperature, are intended to be embraced therein.

1. An Ice Belt To Reduce Body Temperature, comprising:
a strip of flexible material of sufficient length and width to gird the waist from the midriff down, said strip having a body contacting side and an opposite outwardly facing side and an upper edge and lower edge;
pocket-like receptacles secured to the innermost body contacting side of the Ice Belt for the placement of frozen-flexible ice bags—type blue or treated ice; the said belt further contains;
connecting means to secure the upper and lower edges of the belt together and hold the belt in folded condition with the pocket-like receptacles inside the fold and the strip of flexible material to the outside thereof.

2. The belt of claim 1, containing pocket-like receptacles attached to a strip of flexible material for the placement of ice bag's—type blue or treated ice—furthermore contains:
fastening means on the inner side of the upper portion of the pocket-like receptacles; thus securing the ice bags from falling out, and facilitating the removal of the ice bags at intervals for refreezing when the ice melts; depending on the temperature, body functions, and outer garment of the user while wearing the Ice Belt.
